(19) United States
(12) Patent Application Publication
(10) Pub. No.: US 2021/0330801 A1
(43) Pub. Date: Oct. 28, 2021

(54) NOVEL PROTEIN DRUG CONJUGATE FORMULATION

(71) Applicant: CADILA HEALTHCARE LIMITED, Ahmedabad, Gujarat (IN)

(72) Inventors: Sanjeev Kumar MENDIRATTA, Ahmedabad, Gujarat (IN); Sanjay BANDYOPADHYAY, Ahmedabad, Gujarat (IN); Avanish Kumar SINGH, Ahmedabad, Gujarat (IN); Chintan PATEL, Ahmedabad, Gujarat (IN)

(21) Appl. No.: 16/490,208

(22) PCT Filed: Mar. 1, 2018
(86) PCT No.: PCT/IB2018/051302

§ 371 Date: Aug. 30, 2019

(30) Foreign Application Priority Data
Mar. 2, 2017 (IN) 201721007368

(51) Int. Cl. A61K 47/68; C07K 16/32; A61K 47/02; A61K 47/26

(52) U.S. Cl. CPC A61K 47/6803; C07K 16/32; A61K 47/6855; A61K 47/26; A61K 47/02

(57) ABSTRACT

The invention provides stable pharmaceutical formulation comprising a protein drug conjugate along with one or more suitable excipient(s) such that the formulation is devoid of any buffer components and methods of making the same. The protein drug conjugate according to the present invention is antibody drug conjugate, preferably trastuzumab maytansinoid conjugate. Suitable excipient(s) according to the present invention is selected from suitable bulking agents, suitable tonicity modifiers, suitable stabilizers and the like.

### FIELD OF THE INVENTION

The invention provides stable pharmaceutical formulation comprising a protein drug conjugate along with one or more suitable excipient(s) such that the formulation is devoid of any buffer components and methods of making the same. The protein drug conjugate according to the present invention is antibody drug conjugate, preferably trastuzumab maytansinoid conjugate. Suitable excipient(s) according to the present invention is selected from suitable bulking agents, suitable tonicity modifiers, suitable stabilizers and the like.

### BACKGROUND OF THE INVENTION

Protein drug conjugates are developed as highly potent and specific agents for the treatment of cancer and other conditions. A protein drug conjugate is composed of a protein specifically recognizing a target cell antigen, such as a tumor cell antigen, and one or several covalently linked molecules of a drug, particularly a cytotoxic drug such as a maytansinoid, a taxane, or a CC-1065 analog and the like. Protein drug conjugates are inactive during circulation but bind to target cell surfaces, whereupon they are internalized by the cells. By mechanisms not yet fully understood, the drugs are subsequently released from the conjugate and can exert their pharmacological effect.

The targeted delivery of cytotoxic drugs to target cells, such as cells making up cancer tissue, potentially improves the therapeutic indexes of the cytotoxic drugs. Typically, cytotoxic drugs used as protein drug conjugates are 100 to 1000-fold more potent than conventional chemotherapy drugs. These protein drug conjugates are generally combined with one or more pharmaceutically acceptable carriers, excipients, and/or stabilizers to provide a pharmaceutical composition that allows for administration to patients and for storage and transport of the pharmaceutical compound. Like other protein pharmaceuticals, protein drug conjugates are prone to degradation through oxidation, deamidation, as well as particle and aggregate formation. Particle formation in protein pharmaceuticals, in particular, can destabilize the pharmaceutical compound, thus making the formulation less potent or even harmful for clinical use. For example, particles in injected pharmaceutical formulations can cause significant injury to veins or prolonged venous stasis in patients. In addition, aggregate formation is a major degradation pathway of protein pharmaceuticals (Chari et. al., Pharm Res. 20, 1325-1336 (2003)), and may lead to undesirable effects such as immunogenicity.

The conjugation of drugs, especially cytotoxic drugs, which are often hydrophobic small molecules, to hydrophilic monoclonal antibodies, introduces additional instability to protein drug conjugates. Addressing the properties attributable to the protein component of protein drug conjugates is critical to the generation of stable liquid or lyophilized pharmaceutical formulations. Thus, there remains a need for pharmaceutical compositions of protein drug conjugates that are substantially free of particles and/or aggregates, and remain substantially free of particles and/or aggregates during storage and transport.

The present invention provides pharmaceutical compositions of protein drug conjugates that are substantially free of particles and/or aggregates and prevent the formation of particles and/or aggregates during storage and/or transport. Methods for use of the pharmaceutical compositions are also provided. These and other advantages of the invention, as well as additional inventive features, will be apparent from the descriptions of the invention provided herein.

WO 2004/004639 discloses formulation of immunoconjugate that comprises of a buffer (succinic acid and sucrose) to maintain the pH between pH 5.8 to pH 6.2. However, these compositions do not adequately address particle and aggregate formation in pharmaceutical compositions of protein drug conjugates.

US 2002/001587 describe formulation of anti-ErbB-maytansinoid conjugate comprising the said conjugate along with sucrose, polysorbate 20 and 10 nM sodium succinate as buffer at pH 5.0.

WO 2003/105894 describes liquid formulations of SYNAGIS (Palivizumab) or an antigen-binding fragment thererof which comprises 25 mM histidine and 1.6 mM glycine as buffer.

WO 1997/04801 describes lyophilized protein formulations. It also describes stability screening of several lyophilized formulations of recombinant humanized anti-HER 2 antibody, including a formulation comprising 10 nM histidine, 29.2 mM sucrose, 266.4 mM glycine and 0.01% Tween 20.

None of these documents discloses formulation of protein drug conjugates without a buffering agent. Present invention provides stable formulations of protein drug conjugates which do not comprise any buffering agent.

### SUMMARY OF THE INVENTION

in one aspect, the formulation comprising protein drug conjugate with_{—} one or more suitable excipient(s).

In one aspect, the formulation comprises of a protein drug conjugate in water.

In another aspect, the formulation comprising protein drug conjugate with one or more suitable excipient(s) selected from suitable bulking agents, suitable tonicity modifiers, suitable stabilizers and the like.

In another aspect of the invention, the composition of protein drug conjugate further comprises one or more non-ionizable excipient(s).

In a further aspect, the composition of protein drug conjugate of the present invention may further comprise optional ioinic excipients such as tonicity agent(s).

In another aspect, the invention provides a composition of protein drug conjugates which does not contain a buffer.

In one aspect, the formulation comprising the protein drug conjugate which is formulated in water and maintains the stability, during long-term liquid storage and also maintains its integrity during the events of freeze/thaw and lyophilization.

In further aspect, such formulations can also be, optionally lyophilized, Lyophilization can be performed by a skilled person using the techniques available in the art, which includes various steps like freezing, annealing, primary drying and secondary drying.

In one of the aspects, the present invention provides methods and compositions for protein drug conjucate formulations which comprise water and the protein drug conjugate, where the protein drug conjugate is stable without any buffered solution or butler components.

In a still further aspect, the formulation of the invention has improved stability, such as, but not limited to, stability in a liquid form for an extended time for e.g., at least about 3 months or at least about 12 months or maintains stability through at least one freeze/thaw cycle.

In furthermore aspect, the formulation according to the present invention is stable for at least about 3 months in a form selected from the group consisting of frozen or lyophilized.

In a preferred aspect, the protein drug conjugate is trastuzumab maytansinoid conjugates. In a specific embodiment, the trastuzumab maytansinoid conjugate is T-DM1 or trastuzumab emtansine.

In a preferred aspect, the formulation of T-DM1 is in water.

The formulation of the invention may be suitable for any use, including both in vitro and in vh'o uses. In one embodiment, the formulation of the invention is suitable for administration to a subject via a mode of administration, including, but not limited to, subcutaneous, intravenous, intradermal, transdermal, intraperitoneal, and intramuscular administration. The formulation of the invention may be used in the treatment of a disease or disorder in a subject.

### BRIEF DESCRIPTION OF DRAWINGS

FIG. 1: Polypeptide profile of T-DM1 conjugate after 1^{st} and 5^{th} Freeze-Thaw cycle in reducing as well as non-reducing condition

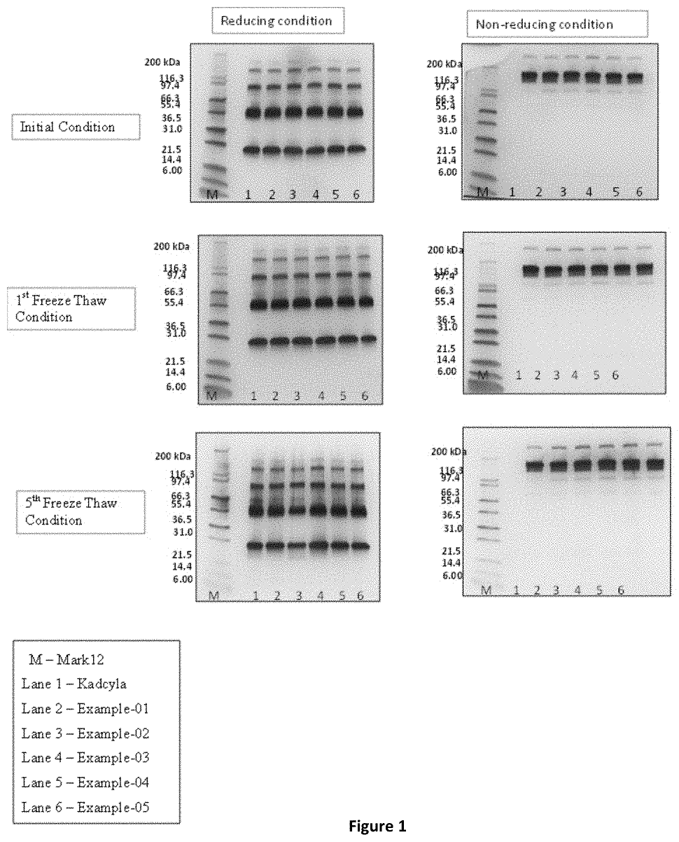

### DETAILED DESCRIPTION OF THE PRESENT INVENTION

The present invention provides novel stable formulations of protein drug conjugates, which can optionally be lyophilized, comprising of suitable amount of therapeutic protein drug conjugates.

In one embodiment of the invention, the protein drug conjugate is an antibody drug conjugate.

In preferred embodiment, the antibody drug conjugate is trastuzumab maytansinoid conjugate, more preferably T-DM1. The drug T-DM1 is commercially being marketed as Kadcyla®.

In an embodiment, the aqueous pharmaceutical formulation comprising essentially of trastuzumab maytansinoid conjugate and water.

In one of the embodiments of the invention, the formulation according to the present invention may further comprise a non-ionizable excipient(s). Examples of non-ionizable excipients include, but are not limited to, a sugar alcohol or polyol (e.g., mannitol or sorbitol), a non-ionic surfactant (e.g., polysorbate 80, polysorbate 20, polysorbate 40, polysorbate 60), and/or a sugar (e.g., sucrose) and suitable combination thereof. Other non-limiting examples of non-ionizable excipients that may be further included in the formulation of the invention include, but are not limited to, trehalose, raffinose, and maltose.

In one embodiment, the present invention provides formulations of trastuzumab maytansinoid conjugate which have typical shelf life about 1 to 5 years, preferably 1 to 4 years, more preferably 2 to 4 years, when stored between 2-8° C. In another embodiment, the present invention provides formulations of trastuzumab maytansinoid conjugate which does not contain any buffering agents.

In another embodiment, the formulations of the invention are stable following at least one freeze/thaw cycles of the formulation, preferably at least three freeze/thaw cycles, more preferably at least five freeze/thaw cycles.

In another embodiment of the invention, the formulation according to the present invention may further optionally comprise one or more other suitable excipients.

In another embodiment of the invention, the formulations according to the present invention further comprise use of PEG (Polyethylene Glycol) as a stabilizer. Examples of suitable polyethylene glycols are polyethylene glycols with a molecular weight of about 200 to 20,000 Da. Preferred polyethylene glycols are PEG 4000, PEG 5000, PEG 6000, PEG 8000, and PEG 10000.

In one of the embodiments, the present invention provides formulations of trastuzumab maytansinoid conjugates comprising a therapeutically effective amount of trastuzumab maytansinoid conjugate and suitable bulking agents selected from sugars such as, but not limited to trehalose, sucrose and the like.

In one of the embodiments, the present invention provides formulations of trastuzumab maytansinoid conjugate comprising a therapeutically effective amount of trastuzumab maytansinoid conjugate and suitable surfactant such as polysorbate 20, polysorbate 80 and the like.

In another embodiment, the present invention provides formulations of trastuzumab maytansinoid conjugate comprising a therapeutically effective amount of trastuzumab maytansinoid conjugates, suitable bulking agents such as trehalose or sucrose and suitable surfactants such as polysorbate.

In yet another embodiment, the formulations of the present invention optionally comprises suitable tonicity modifiers such as sodium chloride, potassium chloride, potassium sulfate or sodium sulfate.

In one of the embodiments, the composition of the present invention is having a pH of about 4 to 8.

In one of the embodiments, the invention is directed towards trastuzumab maytansinoid conjugates formulated in water under appropriate conditions. Such formulations maintain stability and other desired characteristics, during long-term liquid storage or during carrying out of other processing steps, such as freeze/thaw and lyophilization.

In further embodiment, formulations according to the present invention can also be lyophilized. Lyophilization can be performed by a skilled person using the techniques available in the art, which includes various steps like freezing, annealing, primary drying and secondary drying. Such lyophilized formulation can be reconstituted using the techniques known in the art. The resulting lyophilisate after lyophilisation is dissolved with an appropriate amount of reconstitution solution and can then be used either as an injection solution directly or as an additive for an infusion solution. In the case of use as an additive to infusion solutions, the lyophilisate can be dissolved in typically contacted with about 10 ml of a reconstitution solution and a physiological saline solution (0.9% NaCl) was added 250 ml. The resulting infusion solution is then administered usually within about 30 minutes the patient.

The formulations of the invention may be suitable for any use, including both in vitro and in vivo uses. In one embodiment, the formulations of the invention is suitable for administration to a subject via a mode of administration, including, hut not limited to, subcutaneous, intravenous, intradermal, transdermal, intraperitoneal, and intramuscular administration. The formulations of the invention may be used in the treatment of a disorder in a subject.

Also included in the invention are devices that may be used to deliver the formulation of the invention. Examples of such devices include, but are not limited to, a syringe, a pen, an implant, a needle-free injection device and a patch.

In another embodiment, the present invention provides a method of preparing an aqueous formulation comprising a trastuzumab maytansinoid conjugate, preferably trastuzumab emtansine and water, the method comprising providing the trastuzurnab maytansinoid conjugate in a first solution, and subjecting the first solution to diafiltration using water as a diafiltration medium till suitable amount of exchange with the water has been achieved to obtain diafiltered Trastuzumab maytansinoid conjugate solution and thereby prepare the aqueous formulation. In one embodiment, the trastuzumab emtansine in the resulting formulation retains its biological activity.

In a preferred embodiment, the diafiltered trastuzumab maytansinoid conjugate solution is T-DM1 solution.

In one embodiment, the diafiltration medium consists of water.

In one embodiment, the first protein solution is obtained from a mammalian cell expression system that has been purified to remove host cell proteins (HCPs).

In one embodiment, the method of the invention further comprises adding an excipient to the aqueous formulation so obtained through diafiltration.

### Definitions

In order that the present invention may be more readily understood, certain terms are first defined.

The term “pharmaceutical formulation” refers to preparations which are in such form as to permit the biological activity of the active ingredients to be unequivocally effective, and which contain no additional components which are significantly toxic to the subjects to which the formulation would be administered. “Pharmaceutically acceptable” excipients (vehicles, additives) are those which can reasonably be administered to a subject mammal to provide an effective dose of the active ingredient employed.

In a pharmacological sense, in the context of the present invention, a “therapeutically effective amount” or “effective amount” of a protein drug conjugate refers to an amount effective in the prevention or treatment of a disorder for the treatment of which the protein drug conjugate is effective. A “disorder” is any condition that would benefit from treatment with the antibody. This includes chronic and acute disorders or diseases including those pathological conditions which predisposes the subject to the disorder in question.

The term “aqueous formulation” refers to a solution in which the solvent is water.

As used herein, the term “bulking agent” is intended to mean a compound used to add bulk to the reconstitutable solid and/or assist in the control of the properties of the formulation during preparation. Such compounds include, by way of example and without dextran, trehalose, sucrose, polyvinylpyrrolidone, lactose, inositol, sorbitol, dimethylsulfoxide, glycerol, albumin, calcium lactobionate, and others known to those of ordinary skill in the art.

The term “stabilizers” as used herein generally includes agents, which provide stability to the protein from freezing-induced stresses. Examples of stabilizers include polyols such as, for example, mannitol, and include saccharides such as, for example, sucrose, as well as including surfactants such as, for example, polysorbate, poloxamer or polyethylene glycol, and the like. Cryoprotectants also contribute to the tonicity of the formulations.

The term “pharmaceutical” as used herein is with reference to a composition, e.g., an aqueous formulation that it is useful for treating a disease or disorder.

The term “excipient” refers to an agent that may be added to a formulation to provide a desired consistency, (e.g., altering the bulk properties), to improve stability, and/or to adjust osmolality. Examples of commonly used excipients include, but are not limited to, sugars, polyols, amino acids, surfactants, and polymers.

The term “ionic excipient” or “ionizable excipient,” as used interchangeably herein, refers to an agent that has a net charge. In one embodiment, the ionic excipient has a net charge under certain formulation conditions, such as pH, Examples of an ionic excipient include, but are not limited to, histidine, arginine, and sodium chloride. The term “non-ionic excipient” or “non-ionizable excipient,” as used interchangeably herein, refers to an agent having no net charge. In one embodiment, the non-ionic excipient has no net charge under certain formulation conditions, such as pH. Examples of non-ionic excipients include, but are not limited to, sugars (e.g., sucrose), sugar alcohols (e.g., mannitol), and non-ionic surfactants (e.g., polysorhate 80),

The phrase “protein is dissolved in water” as used herein refers to a formulation of a protein wherein the protein is dissolved in an aqueous solution in which the amount of small molecules (e.g., buffers, excipients, salts, and surfactants) has been reduced by DF/UF processing. Even though the total elimination of small molecules cannot be achieved in an absolute sense by DF/UF processing, the theoretical reduction of excipients achievable by applying DF/UF is sufficiently large to create a formulation of the protein essentially in water exclusively. The term “surfactants” generally includes those agents that protect the protein from air/solution interface-induced stresses and solution/surface induced-stresses. For example surfactants may protect the protein from aggregation, Suitable surfactants may include, e.g., polysorbates, polyoxyethylene alkyl ethers such as Brij 35®, or poloxamer such as Tween 20, Tween 80, or poloxamer 188. Preferred detergents are poloxamers, e.g., Poloxamer 188, Poloxamer 407; polyoxyethylene alkyl ethers, e.g., Brij 35®, Cremophor A25, Sympatens ALM/230; and polysorbates/Tweens, e.g., Polysorbate 20, Polysorbate 80, and Poloxamers, Poloxamer 188, and Tweens, e.g., Tween 20 and Tween 80.

As used herein, the term “tonicity modifier” is intended to mean a compound or compounds that can be used to adjust the tonicity of a liquid formulation. Suitable tonicity modifiers include glycerin, lactose, mannitol, dextrose, sodium chloride, magnesium sulfate, magnesium chloride, sodium sulfate, sorbitol, trehalose, sucrose, raffinose, maltose and others known to those or ordinary skill in the art. In one embodiment, the tonicity of the liquid formulation approximates that of the tonicity of blood or plasma.

The term “water” is intended to mean water that has been purified to remove contaminants, usually by distillation or reverse osmosis, also referred to herein as “pure water”. In a preferred embodiment, water used in the methods and compositions of the invention is excipient-free. In one embodiment, water includes sterile water suitable for administration to a subject. In another embodiment, water is meant to include)1 injection (WH). In one embodiment, water refers to distilled water or water which is appropriate for use in in vitro assays. In a preferred embodiment, diafiltration is performed in accordance with the methods of the invention using water alone as the diafiltration medium.

The term “protein drug conjugate” refers to conjugate of protein to a drug, optionally via linker. It is well defined in the art for example, U.S. Pat. No. 4,981,979, U.S. Pat. No. 5,208,020, WO 1997020858, WO 2002/057316, WO 2005001038, WO0243661, WO2004010957, WO2005001038, WO2004010957 WO2005001038 and many other patent and non-patent literatures disclosing conjugation of protein with drug.

Examples of such protein drug conjugates are conjugates of antibodies such as ErbB receptor targeting antibodies, preferably anti-HER2 or anti-1-IER3 targeting antibody with cytotoxic drug such as maytansinoid, taxa auristatin, conjugates of proteins or peptides such as hormones, trasnferrins, lipocalins with cytotoxic drug such as maytansinoid, taxol, auristatin, etc. Components of protein drug conjugates are defined herein below but this definition is non-limiting to the present invention.

The trastuzumab maytansinoid conjugate is prepared as per the process as mentioned in Indian patent application 20172101491 7 and WO 2001/000244.

The term “T-DM1” is trastuzumab maytansinoid conjugate which is known as trastuzumab emtansine. Trastuzumab emtansine (it can be referred as T-DM1 or trastuzumab-MCC-DM1) is the trastuzumab antibody covalently bound to DM1. In T-DM1, this antibody is linked to a hetero-bifunctional reagent, succinimidyl trans-4-[maleimidylmethyl] cyclohexane-l-carboxylate (SMCC). The other end of the SMCC linker molecule is covalently bound to DM1 by a labile thioether bond. The antibody binds to the linker predominantly at lysine residues with a net stoichiometry of DM1 to antibody of approximately 3.5. The resulting compound is referred to as an antibody drug conjugate or ADC.

Here, the protein according to the present invention is a cell-binding agent.

### Cell-Binding Agents

The effectiveness of the compounds of the invention as therapeutic agents depends on the careful selection of an appropriate cell-binding agent. Cell-binding agents may be of any kind presently known, or that become known and includes peptides and non-peptides. Generally, these can be antibodies (especially monoclonal antibodies), lymphokines, hormones, growth factors, vitamins, nutrient-transport molecules (such as transferrin), or any other cell-binding molecule or substance that specifically binds a target. More specific examples of cell-binding agents that can be used include: polyclonal and monoclonal antibodies, including fully human antibodies; single chain antibodies (polyclonal and monoclonal); fragments of antibodies (polyclonal and monoclonal) such as Fab, Fab′,

F(ab′)₂, and Fv., chimeric antibodies and antigen-binding fragments thereof; domain antibodies (dAbs) and antigen-binding fragments thereof, including camelid antibodies, shark antibodies called new antigen receptors (IgNAR) interferons (e.g. alpha, beta, gamma); lymphokines such as IL-2, IL-3, IL-4, 1L-6; hormones such as insulin, TRH (thyrotropin releasing hormone), MSH (melanocyte-stimulating hormone), steroid hormones, such as androgens and estrogens; growth factors and colony-stimulating factors such as EGF, TGF-alpha, FGF, VEGF, G-CSF, M-CSF and GM-CSF, transferrin, human tear lipocalin or its muteins and vitamins, such as folate.

The term “linker” refers to any chemical moiety that links a cell-binding agent covalently to a drug. In some instances, part of the linker is provided by the drug. Therefore the final linker is assembled from two pieces, the cross-linking reagent introduced into the cell-binding agent and the side chain from the drug. Linkers may broadly be either a cleavable linker or a non-cleavable linker.

Cleavable linkers are linkers that can be cleaved under mild conditions, i.e. conditions under which the activity of the maytansinoid drug is not affected. Many known linkers fall in this category and are described below:)
i) Disulfide containing linkers are linkers cleavable through disulfide exchange, which can occur under physiological conditions.
ii) Acid-labile linkers are linkers cleavable at acid pH. For example, certain intracellular compartments, such as endosomes and lysosomes, have an acidic pH (pH 4-5), and provide conditions suitable to cleave acid-labile linkers.
iii) Linkers that are photo-labile are useful at the body surface and in many body cavities that are accessible to light. Furthermore, infrared light can penetrate tissue. Some linkers can be cleaved by peptidases. Only certain peptides are readily cleaved inside or outside cells, see e.g. Trouet et al., 79 Proc. Nail Acad. Sci. USA, 626-629 (1982) and limernoto et al. 43 Int. I Cancer, 677-684 (1989). Furthermore, peptides are composed of α-amino acids and peptidic bonds, which chemically are amide bonds between the carboxylate of one amino acid and the α-amino group of a second amino acid. Other amide bonds, such as the bond between a carboxylate and the ε-amino group of lysine, are understood not to be peptidic bonds and are considered non-cleavable.
iv) Some linkers can be cleaved by esterases. Again only certain esters can be cleaved by esterases present inside or outside cells. Esters are formed by the condensation of a carboxylic acid and an alcohol. Simple esters are esters produced with simple alcohols, such as aliphatic alcohols, and small cyclic and small aromatic alcohols.

A non-cleavable linker is any chemical moiety that is capable of linking a maytansinoid to a cell-binding agent in a stable, covalent manner and does not fall under the categories listed above as cleavable linkers. Thus, non-cleavable linkers are substantially resistant to acid-induced cleavage, light-induced cleavage, peptidase-induced cleavage, esterase-induced cleavage, and disulfide bond cleavage.

“Substantially resistant” to cleavage means that the chemical bond in the linker or adjoining the linker in at least 80%, preferably at least 85%, more preferably at least 90%, even more preferably at least 95%, and most preferably at least 99% of the cell-binding agent maytansinoid conjugate population remains non-cleavable by an acid, a photolabile-cleaving agent, a peptidase, an esterase, or a chemical or a physiological compound that cleaves the chemical bond (such as a disulfide bond) in a cleavable linker, for within a few hours to several days of treatment with any of the agents described above.

Furthermore, “non-cleavable” refers to the ability of the chemical bond in the linker or adjoining to the linker to withstand cleavage induced by an acid, a photolabile-cleaving agent, a peptidase, an esterase, or a chemical or a physiological compound that cleaves a disulfide bond, at conditions under which the maytansinoid or the cell binding agent does not lose its activity.

A person of ordinary skill in the art would readily distinguish non-cleavable from cleavable linkers.

### Drug

Suitable drugs may include radionuclides, toxins, small organic molecules, and therapeutic peptides (such as peptides acting as agonists/antagonists of a cell surface receptor or peptides competing for a protein binding site on a given cellular target). Examples of suitable toxins include, but are not limited to pertussis-toxin, diphtheria toxin, ricin, saporin, pseudomonas exotoxin, calicheamicin or a derivative thereof, a taxoid, a maytansinoid, a tuhulysin or a dolastatin analogue. The dolastatin analogue may be auristatin E., inonomethylauristatin F., auristatin PYE and auristatin. PHE. Examples of cytostatic agent include, but are not limited to Cisplatin, Carhop Oxaliplatin, 5-Fluorouracil, Taxotere (Docetaxel), Paclitaxel, Anthracycline (Doxorubicin), Methotrexate, Vinblastin, Vincristine, Vindesines, Vinorelbi ne, Dacarbazine, Cyclophosphamide, Etoposide, Adriamycine, Camptotecine, Cornbretata.stin A-4 related compounds, sulfonamides, oxadiazolines, benzo[b]thiophenessynthetic spiroketal pyrans, monotetrahydrofuran compounds, curacin and curacin derivatives, methoxyestradiol derivatives, CC-1065 ,CC-1065 analogs and Leucovorin.

As used herein, the terms “ultrafiltration” or “UF” refers to any technique in which a solution or a suspension is subjected to a semi-permeable membrane that retains macromolecules while allowing solvent and small solute molecules to pass through.

Ultrafiltration may be used to increase the concentration of macromolecules in a solution or suspension. In a preferred embodiment, ultrafiltration is used to increase the concentration of a protein in water.

As used herein, the term “diafiltration” or “DF” is used to mean a specialized class of filtration in which the retentate is diluted with solvent and re-filtered, to reduce the concentration of soluble permeate components. Diafiltration may or may not lead to an increase in the concentration of retained components, including, for example, proteins. For example, in continuous diafiltration, a solvent is continuously added to the retentate at the same rate as the filtrate is generated. In this case, the retentate volume and the concentration of retained components do not change during the process. On the other hand, in discontinuous or sequential dilution diafiltration, an ultrafiltration step is followed by the addition of solvent to the retentate side; if the volume of solvent added to the retentate side is not equal or greater to the volume of filtrate generated, then the retained components will have a high concentration. Diafiltration may be used to alter the pH, ionic strength, salt composition, buffer composition, or other properties of a solution or suspension of macromolecules.

As used herein, the terms “diafiltration/ultrafiltration” or “DF/Uf” refer to any process, technique or combination of techniques that accomplishes ultrafiltration and/or diafiltration, either sequentially or simultaneously.

As used herein, the term “first protein solution” or “first solution” refers to the initial protein solution or starting material used in the methods of the invention, i.e., the initial protein solution which is diafiltered into water. In one embodiment, the first protein solution comprises ionic excipients, non-ionic excipients, and/or a buffering system.

As used herein, the term “diafiltration step” refers to a total volume exchange during the process of diafiltration,

### EXAMPLES

The following non-limiting examples describe the different formulations which can be prepared as per the present invention, it will be appreciated that other excipients may be added as are necessary to these formulations and such addition of excipients are within the scope of a person skilled in the art and are to be included within the scope of the present invention,

The following examples describe experiments relating to an aqueous formulation comprising water as the solution medium. These formulations can optionally be lyophilized using techniques known in the art.

### Examples 1

### Diafiltration/Ultrafiltration of Trastuzumab Emtansine (T-DM1)

Trastuzumab emtansine was prepared as described in WO 2001/000244 and in

Indian patent application 201721014917.

TABLE 1

| T-DM1 | 20 mg/mL |
|---|---|
| WFI | — |
| pH | 6.1 |

T-DM1 solution was brought into the WFI medium through ultrafiltration/diafiltration by using 30 kDa MWCO membrane filter. After ultrafiltration/diafiltration, concentration of T-DM1 was adjusted to about 20 mg/mL. After the buffer exchange step, the concentrated purified T-DM1 solution was filtered through a 0.22 pm filter, under aseptic conditions.

Osmolality determination, pH, DAR (Drug to Antibody Ratio), and T-DM1 concentration measurements (OD280) were performed to monitor the status of the T-DM1 during DF/UF processing. Data of the initial analysis are given in table-6, data after 1^{st} freeze thaw condition are given in table-7 and data after 5^{th} freeze thaw condition are given in table-8.

### Examples 2

### T-DM1 with Bulking Agent (Trehalose)

TABLE 2

| | | |
|---|---|---|
| T-DM1 | 20 | mg/mL |
| Trehalose | 19.09 | mg/mL |

Diafiltered T-DM1 of Example 1 was formulated using trehalose as mentioned in Table 2. Osmolality determination Conductance, pH, DAR (Drug to Antibody Ratio), and T-DM1 concentration measurements (OD280) were performed to monitor the status of the T-DM1. Data of the initial analysis are given in table-6, data after 1^{st} freeze thaw condition are given in table-7 and data after 5^{th} freeze thaw condition are given in table-8.

### Examples 3

### T-DM1 with Surfactant and Bulking Agent

TABLE 3

| | | |
|---|---|---|
| T-DM1 | 20 | mg/mL |
| Trehalose | 19.09 | mg/mL |
| Polysorbate 20 | 0.084 | mg/mL |

Diafiltered T-DM1 was formulated along with Trehalose and Polysorbate 20 as per Table 3 above. Osmolality determination Conductance, pH, DAR (Drug to Antibody Ratio), and T-DM1 concentration measurements (0D280) were performed to monitor the status of theT-DM1. Data of the initial analysis are given in table-6, data after 1^{st} freeze thaw condition are given in table-7 and data after 5 freeze thaw condition are given in table-8.

### Example 4

### T-DM1 with Tonicity Modifier and Bulking Agent

TABLE 4

| | | |
|---|---|---|
| T-DM1 | 20 | mg/mL |
| Trehalose | 19.09 | mg/mL |
| Sodium chloride | 100 | mM |

Diafiltered T-DM1 was formulated as mentioned in as per Table 4 above. Osmolality determination Conductance, p1-1. DAR (Drug to Antibody Ratio), and T-DM1 concentration measurements (OD280) were performed to monitor the status of the T-DM1 , Data of the initial analysis are given in table-6.

### Example 5

### T-DM1 with Tonicity Modifier, Surfactant and Bulking Agent

TABLE 5

| | | |
|---|---|---|
| T-DM1 | 20 | mg/mL |
| Trehalose | 19.09 | mg/mL |
| Polysorbate 20 | 0.084 | mg/mL |
| Sodium chloride | 100 | mM |

Diafiltered T-DM1 was formulated as per Table 5 above. Osmolality determination Conductance, pH, DAR (Drug to Antibody Ratio), and T-DM1 concentration measurements (OD280) were performed to monitor the status of the T-DM1 . Data of the initial analysis are given in table-6.

TABLE 6
Result of analysis done for formulations as described in examples 1 to 5

| | | | | Osmolality | Purity analysis by HP-SEC | | |
|---|---|---|---|---|---|---|---|
| Sample | | Concentration | | (mOsm/kg | % HMW | % Principal | % LMW |
| description | pH | (mg/mL) | DAR | of water) | species | peak | species |
| Example 1 | 6.1 | 19.98 | 3.85 | 1 | 1.77 | 98.23 | bq |
| Example 2 | 6.2 | 20.45 | 3.90 | 48 | 1.79 | 98.21 | bq |
| Example 3 | 6.0 | 20.24 | 3.85 | 53 | 1.77 | 98.23 | bq |
| Example 4 | 6.0 | 18.62 | 3.78 | 306 | 1.85 | 98.15 | bq |
| Example 5 | 6.3 | 18.33 | 3.81 | 254 | 1.82 | 98.18 | bq |
| Trastuzumab | 6.1 | 25.86 | 3.83 | 1 | 1.75 | 98.25 | bq |
| maytansinoid | | | | | | | |
| conjugate | | | | | | | |
| concentration | | | | | | | |
| (After UF/DF) | | | | | | | |

bq: Below quantitation

TABLE 7
Result of analysis done after 1^{st} Freeze thaw for
formulations as described in examples 1 to 3

| | | | | | Purity analysis by HP-SEC | | |
|---|---|---|---|---|---|---|---|
| Sample | | Concentration | | Osmolality | % HMW | % Principal | % LMW |
| description | pH | (mg/mL) | DAR | (mOsm/kg) | species | peak | species |
| Example 1 | 5.99 | 21.89 | 3.25 | 14 | 2.01 | 97.99 | bq |
| Example 2 | 5.9 | 20.30 | 3.21 | 53 | 1.68 | 98.32 | bq |
| Example 3 | 5.9 | 19.95 | 3.16 | 53 | 1.70 | 98.30 | bq |

TABLE 8
Result of analysis done after 5^{th} Freeze thaw for
formulations as described in examples 1 to 3

| | | | | | Purity analysis by HP-SEC | | |
|---|---|---|---|---|---|---|---|
| Sample | | Concentration | | Osmolality | % HMW | % Principal | % LMW |
| description | pH | (mg/mL) | DAR | (mOsm/kg) | species | peak | species |
| Example 1 | 5.99 | 19.48 | 3.18 | 0 | 2.55 | 97.45 | bq |
| Example 2 | 5.86 | 19.50 | 3.25 | 49 | 1.87 | 98.13 | bq |
| Example 3 | 5.85 | 19.04 | 3.26 | 49 | 1.85 | 98.15 | bq |

The results of Tables 7 & 8 indicate that the formulations of the current invention remains stable even after five freeze thaw cycles.

The aqueous pharmaceutical formulations of the present invention have a shelf life of at least 1 year when stored between 2-8° C.

1. An aqueous pharmaceutical formulation comprising essentially of :
a. Trastuzumab maytansinoid conjugate and
b. Water.

2. An aqueous pharmaceutical formulation comprising:
a. Trastuzumab maytansinoid conjugate; and
b. water;
wherein the formulation does not contain buffering agent.

3. The aqueous pharmaceutical formulation as claimed in claim 2 which further comprises one or more non-ionizable excipients.

4. The aqueous pharmaceutical formulation as claimed in claim 3, wherein the non-ionizable excipient is selected from sugar alcohol, polyol, a non-ionic surfactant, sugar and suitable combination thereof.

5. The aqueous pharmaceutical formulation as claimed in claim 4, wherein polyol is selected from mannitol or sorbitol.

6. The aqueous pharmaceutical formulation as claimed in claim 4, wherein the non-ionic surfactant is selected from polysorbate 80, polysorbate 20, polysorbate 40, polysorbate 60.

7. The aqueous pharmaceutical formulation as claimed in claim 4, wherein sugar is selected from sucrose, trehalose, raffinose and maltose.

8. The aqueous pharmaceutical formulation as claimed in claim 2 which further comprises one or more tonicity modifiers.

9. The aqueous pharmaceutical formulation as claimed in claim 8, wherein tonicity modifier is selected from sodium chloride, potassium chloride, potassium sulfate and sodium sulfate.

10. The aqueous pharmaceutical formulation as claimed in claim 2 wherein the pH of the formulation is between pH 4 to pH 8.

11. The aqueous pharmaceutical formulation as claimed in claim 1 2 having a shelf life of 1 to 5 years, when stored between 2-8° C.

12. The aqueous pharmaceutical formulation as claimed in claim 2 which is stable for at least one freeze/thaw cycle.

13. The aqueous pharmaceutical formulation as claimed in claim 2 which maintains stability during long-term liquid storage or during carrying out of other processing steps selected from freeze/thaw and lyophilization.

14. The aqueous pharmaceutical formulation as claimed in claim 2 which is suitable for administration to a subject via a mode of administration including subcutaneous, intravenous, intradermal, transdermal, intraperitoneal and intramuscular administration.

15. A lyophilized formulation comprising the composition as claimed in claim 2.

16. A method of preparing formulation of trastuzumab maytansinoid conjugate as claimed in claim 2 comprising;
a. Providing the Trastuzumab maytansinoid conjugate in a first solution;
b. Subjecting the first solution to diafiltration using diafiltration medium till suitable amount of exchange with diafiltration medium has been achieved to obtain diafiltered Trastuzumab maytansinoid conjugate solution, wherein diafiltration medium is a buffer or water.

17. The method of preparing formulation of trastuzumab maytansinoid conjugate as claimed in claim 16 wherein diafiltration medium is water.

18. The method of preparing formulation of trastuzumab maytansinoid conjugate as claimed in claim 16 wherein the diafiltered Trastuzumab maytansinoid conjugate solution is achieved via ultrafiltration diafiltration.

19. The pharmaceutical formulation of trastuzumab maytansinoid conjugate as claimed in claim 2, wherein trastuzumab maytansinoid conjugate is T-DM1.

20. A lyophilized formulation comprising the composition as claimed in claim 1.
